(19) United States
(12) Patent Application Publication
(10) Pub. No.: US 2024/0287550 A1
(43) Pub. Date: Aug. 29, 2024

(54) SYSTEMS AND METHODS FOR UTILIZING CORN COBS TO JOINTLY PRODUCE PREMIUM XYLOSE AND HIGH-END CARAMEL PIGMENT

(71) Applicant: ZHEJIANG HUAKANG PHARMACEUTICAL CO., LTD., Quzhou (CN)

(72) Inventors: Weidong XU, Quzhou (CN); Mian LI, Quzhou (CN); Wulong YANG, Quzhou (CN); Qiang WU, Quzhou (CN); Shufang QIN, Quzhou (CN); Jingjing XU, Quzhou (CN); Zhihui YAO, Quzhou (CN)

(73) Assignee: ZHEJIANG HUAKANG PHARMACEUTICAL CO., LTD., Quzhou (CN)

(21) Appl. No.: 18/656,572

(22) Filed: May 6, 2024

Related U.S. Application Data
(63) Continuation-in-part of application No. PCT/CN2023/096361, filed on May 25, 2023.

(30) Foreign Application Priority Data
Dec. 5, 2022 (CN) 202211552492.6

(51) Int. Cl. C12P 7/26; C12P 19/02

(52) U.S. Cl. CPC C12P 7/26; C12P 19/02; C12P 2201/00

(57) ABSTRACT

The present disclosure provides a system and a method for utilizing corn cobs to jointly produce a premium xylose and a high-end caramel pigment. The system includes a xylose preparation subsystem, an enzymatic hydrolysis subsystem, and a caramel pigment preparation subsystem that are connected to each other through pipelines. The xylose preparation subsystem includes a raw material tank, a pretreatment tank, an acid hydrolysis kettle, a plate and frame filter press device, a filtrate neutralization tank, a nanofiltration membrane separator, an electrodialysis separator, an evaporation concentration tank, a crystallization tank, a centrifugal separator, and a dryer that are connected in sequence through pipelines. The enzymatic hydrolysis subsystem includes a filter residue neutralization tank and an enzymatic hydrolysis reaction kettle connected through pipelines. The caramel pigment preparation subsystem includes a sugar liquid mixing tank, a browning reaction kettle, a flash tank, an ultrafiltration membrane separator, and a wiped film evaporator.

### CROSS REFERENCE TO RELATED APPLICATIONS

This application is a continuation-in-part of international application No. PCT/CN2023/096361, filed on May 25, 2023, which claims priority to Chinese patent application No. 202211552492.6, filed on Dec. 5, 2022, the entire contents of each of which are incorporated herein by reference.

### TECHNICAL FIELD

The present disclosure relates to the technical field of sugar alcohol preparation, and in particular, to a system and a method for utilizing corn cobs to jointly produce a premium xylose and a high-end caramel pigment.

### BACKGROUND

Xylose is a type of pentose sugar, with a white crystal appearance and a sweet taste, equivalent to 0.7 times the sweetness of sucrose, which can be used as a sucrose substitute for sugar reduction and sweetening purposes. When compared to qualified xylose, premium xylose has a higher purity and light transmittance. High-end caramel pigments refer to those with a high color intensity and high durability, with more than twice the color intensity of regular caramel pigments. Chinese patent CN1111647694A discloses a method for extracting xylose from corn cobs, involving processes such as acid hydrolysis, decolorization, separation, evaporation, and concentration, as well as crystallization and separation, resulting in a finished xylose product. Most of the solid residues of corn cob waste after acid hydrolysis are usually directly incinerated, while the majority of the xylose mother liquid after crystallization is often sold at a low price to syrup manufacturers, leading to significant waste of hexose sugars in the corn cob waste residues and pentose sugars in the xylose mother liquid.

### SUMMARY

Some embodiments of the present disclosure provide a system and a method for utilizing corn cobs to jointly produce a premium xylose and a high-end caramel pigment. The system uses corn cobs as a raw material to extract pentose sugar for the preparation of premium xylose, and processes pentose sugar and hexose sugar from a corn cob residue to produce the high-end caramel pigment. This approach not only reduces the waste of various sugar resources in corn cobs but also converts environmentally harmful agricultural waste into high-value products, thereby promoting high-end manufacturing, environmental and ecological protection, and high-quality economic development.

Some embodiments of the present disclosure provide a system for utilizing corn cobs to jointly produce a premium xylose and a high-end caramel pigment. The system may include a xylose preparation subsystem, an enzymatic hydrolysis subsystem, and a caramel pigment preparation subsystem connected to each other through pipeline. The xylose preparation subsystem may include a raw material tank, a pretreatment tank, an acid hydrolysis kettle, a plate and frame filter press device, a filtrate neutralization tank, a nanofiltration membrane separator, an electrodialysis separator, an evaporation concentration tank, a crystallization tank, a centrifugal separator, and a dryer that are connected in sequence through pipelines. The enzymatic hydrolysis subsystem may include a filter residue neutralization tank and an enzymatic hydrolysis reaction kettle connected through pipelines. The caramel pigment preparation subsystem may include a sugar liquid mixing tank, a browning reaction kettle, a flash tank, an ultrafiltration membrane separator, and a wiped film evaporator connected in sequence through pipelines. The plate and frame filter press device may be provided with a filtrate outlet and a filter residue outlet. The filtrate outlet may be connected to the filtrate neutralization tank through a pipeline. The filter residue outlet may be connected to a feed port of the filter residue neutralization tank through a pipeline. The centrifugal separator may be provided with a solid outlet and a liquid outlet. The solid outlet may be connected to a feed port of the dryer through a pipeline. The liquid outlet may be connected to one feed port of the sugar liquid mixing tank, and the other feed port of the sugar liquid mixing tank may be connected to a discharge port of the enzymatic hydrolysis reaction kettle through a pipeline. The raw material tank may store a raw material to be processed of the corn cobs. A material output by a discharge port of the dryer may be the premium xylose. A final material obtained from a discharge port of the wiped film evaporator may be the high-end caramel pigment.

In some embodiments, a filtrate output from the filtrate outlet of the plate and frame filter press device may be a corn cob hydrolysate, and a filter residue output from the filtrate residue outlet of the plate and frame filter press device may be a corn cob waste residue.

In some embodiments, a liquid material transported from a liquid outlet of the centrifugal separator may be a xylose mother liquid.

In some embodiments, a liquid output from the discharge port of the enzymatic hydrolysis reaction kettle may be an enzymatic solution.

Some embodiments of the present disclosure further provide a method for utilizing corn cobs to jointly produce a premium xylose and a high-end caramel pigment using the system as described in any one of the previous items. The method may include:
Step 1: transporting corn cob powder stored in the raw material tank to the pretreatment tank through a pipeline for water washing and acid washing to obtain a corn cob liquid, and then sending the corn cob liquid to the acid hydrolysis kettle through a pipeline to perform acid hydrolysis treatment;
Step 2: obtaining a plate and frame filter press feed liquid after the acid hydrolysis treatment, and sending the plate and frame filter press feed liquid to the plate and frame filter press device through a pipeline for filtration treatment;
Step 3: filtering the plate and frame filter press feed liquid by the plate and frame filter press device to obtain a liquid part and a solid part, respectively; wherein the liquid part is a corn cob hydrolysate, and the solid part is a corn cob waste residue;
Step 4: performing neutralization treatment of the filtrate neutralization tank, decolorization treatment of the nanofiltration membrane separator, desalination treatment of the electrodialysis separator, concentration treatment of the evaporation concentration tank, crystallization treatment of the crystallization tank, and solid-liquid separation treatment of the centrifugal separator on the corn cob hydrolysate to obtain a crystallized xylose and a xylose mother liquid, respectively, performing drying treatment of the dryer on the crystallized xylose to obtain a crystal finished product of a premium xylose, transporting the xylose mother liquid to the sugar liquid mixing tank through a pipeline; wherein a purity of the crystal finished product of the premium xylose is larger than 99%, and a light transmittance of the crystal finished product of the premium xylose is larger than 98%; and
Step 5: transporting the corn cob waste residue sequentially to the filter residue neutralization tank for neutralization treatment and to the enzymatic hydrolysis reaction kettle for enzymatic hydrolysis treatment to obtain an enzymatic solution, transporting the enzymatic solution to the sugar liquid mixing tank through a pipeline to be mixed with the xylose mother liquid to obtain a mixture solution, and then transporting the mixture solution through browning reaction treatment of the browning reaction kettle, evaporation treatment of the flash tank, separation treatment of the ultrafiltration membrane separator, and wiped film evaporation treatment of the wiped film evaporator to obtain a product of the high-end caramel pigment; wherein a color ratio of the high-end caramel pigment is as high as 50000 EBC.

### BRIEF DESCRIPTION OF THE DRAWINGS

FIG. 1 is a schematic diagram illustrating a principle of a system for utilizing corn cobs to jointly produce a premium xylose and a high-end caramel pigment according to some embodiments of the present disclosure;

FIG. 2 is a schematic diagram illustrating a flow direction of each material in a method for utilizing corn cobs to jointly produce a premium xylose and a high-end caramel pigment according to some embodiments of the present disclosure;

FIG. 3 is a flowchart illustrating an exemplary process for controlling a charging device and a temperature control device according to some embodiments of the present disclosure; and

FIG. 4 is a schematic diagram illustrating a prediction model according to some embodiments of the present disclosure.

### DETAILED DESCRIPTION

In order to make the technical problems, technical solutions, and beneficial effects to be solved by some embodiments of the present disclosure clearer and more understandable, the following combines the accompanying drawings and the embodiments to further explain some embodiments of the present disclosure in detail. It should be understood that the specific embodiments described herein are only for explaining some embodiments of the present disclosure and are not intended to limit some embodiments of the present disclosure.

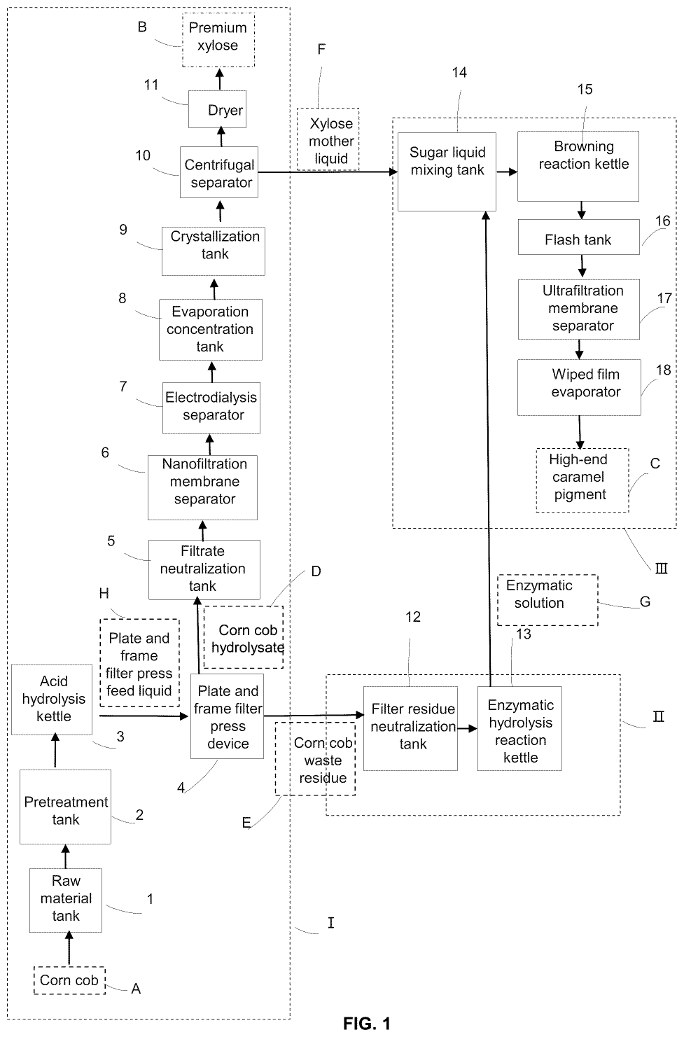

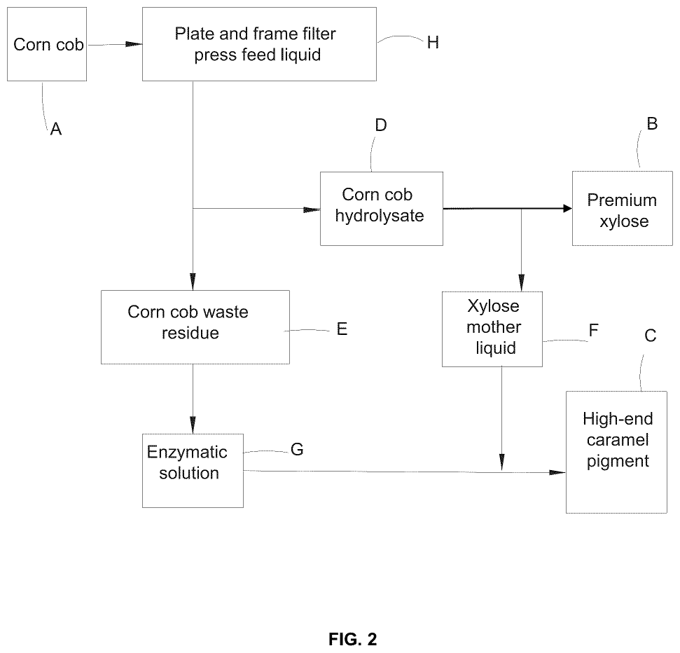

Please also refer to FIG. 1 and FIG. 2, a system for utilizing corn cobs to jointly produce a premium xylose and a high-end caramel pigment in some embodiments of the present disclosure may include a xylose preparation subsystem I, an enzymatic hydrolysis subsystem II, and a caramel pigment preparation subsystem III that are connected to each other by pipelines. The directions of the arrows in the figures illustrate the directions of the flow of materials in the system.

The xylose preparation subsystem I refers to a system for preparing a xylose based on corn cob raw materials. In some embodiments, the xylose preparation subsystem I may include a raw material tank 1, a pretreatment tank 2, an acid hydrolysis kettle 3, a plate and frame filter press device 4, a filtrate neutralization tank 5, a nanofiltration membrane separator 6, an electrodialysis separator 7, an evaporation concentration tank 8, a crystallization tank 9, a centrifugal separator 10, and a dryer 11, which are connected sequentially by pipelines.

The enzymatic hydrolysis subsystem II refers to a system for performing cellulolytic enzymatic hydrolysis on a corn cob waste residue. The enzymatic hydrolysis subsystem II may also be configured to neutralize the corn cob waste residue. In some embodiments, the enzymatic hydrolysis subsystem II may include a filter residue neutralization tank 12 and an enzymatic hydrolysis reaction kettle 13 connected by a pipeline. More descriptions about the filter residue neutralization tank 12 and the enzymatic hydrolysis reaction kettle 13 may be found in the following.

The caramel pigment preparation subsystem III refers to a system for preparing caramel pigments based on the corn cob raw materials. In some embodiments, the caramel pigment preparation subsystem Ill may include a sugar liquid mixing tank 14, a browning reaction kettle 15, a flash tank 16, an ultrafiltration membrane separator 17, and a wiped film evaporator 18 connected sequentially by pipelines.

In some embodiments, the raw material tank 1 may store a raw material to be processed of a corn cob A, a material outputted from a discharge port of the dryer 11 may be a premium xylose B, and a material finally obtained from a discharge port of the wiped film evaporator 18 may be a high-end caramel pigment C.

In some embodiments, the plate and frame filter press device 4 may be provided with a filtrate outlet and a filter residue outlet, the filtrate outlet may be connected to the filtrate neutralization tank 5 through a pipeline, and the filter residue outlet may be connected to a feed port of the filter residue neutralization tank 12 through a pipeline. The filtrate output from the filtrate outlet of the plate and frame filter press device 4 may be a corn cob hydrolysate D, and the filter residue output from the filter residue outlet of the plate and frame filter press device 4 may be a corn cob waste residue E.

In some embodiments, the centrifugal separator 10 may be provided with a solid outlet and a liquid outlet, respectively. The solid outlet may be connected to a feed port of the dryer 11 via a pipeline, and the liquid outlet may be connected to one feed port of the sugar liquid mixing tank 14. The other feed port of the sugar liquid mixing tank 14 may be connected to a discharge port of the enzymatic hydrolysis reaction kettle 13 through a pipeline.

In some embodiments, a liquid material transported from a liquid outlet of the centrifugal separator 10 to the sugar liquid mixing tank 14 may be a xylose mother liquid F. The liquid output from the discharge port of the enzymatic hydrolysis reaction kettle 13 may be an enzymatic solution G.

In some embodiments, the pretreatment tank 2 may be used to wash away impurities in the corn cob A using water or dilute acid, the acid hydrolysis kettle 3 may be used to hydrolyze hemicellulose in the corn cob A, and the plate and frame filter press device 4 may be used to separate the corn cob hydrolysate D from the corn cob waste residue E. The filtrate neutralization tank 5 may be used to neutralize the pH of the corn cob hydrolysate D. The nanofiltration membrane separator 6 may be used for decolorization of the corn cob hydrolysate D. The retentate after decolorization treatment of the nanofiltration membrane separator 6 may be a pigmented liquid, and the permeate may be a decolorized liquid.

The electrodialysis separator 7 may be used for desalination of a decolorized solution. In some embodiments, the retentate after desalination treatment of the electrodialysis separator 7 may be a concentrated saline solution, and the permeate may be a desalinated solution. The evaporation concentration tank 8 and the crystallization tank 9 may be used for evaporation and concentration, and xylose crystallization. The centrifugal separator 10 may be used for separating a crystallized xylose and the xylose mother liquid F, and the dryer 11 may be used for drying a crystal of the premium xylose B.

The filter residue neutralization tank 12 may be used to neutralize the pH of the corn cob waste residue E. The enzymatic hydrolysis reaction kettle 13 may be used to enzymatically hydrolyze the cellulose in the corn cob waste residue E into glucose. The sugar liquid mixing tank 14 may be used for mixing the xylose mother liquid F and the enzymatic solution G to obtain a mixed sugar solution. The browning reaction kettle 15 may be used for browning reaction of the mixed sugar solution to produce a crude high-end caramel pigment. The flash tank 16 may be used to remove impurities such as amino compounds or sulfites in the caramel pigment. The ultrafiltration membrane separator 17 may be used to remove impurities such as 4-methylimidazole in the caramel pigment, and the wiped film evaporator 18 may be used for low-temperature concentration of the caramel pigment to obtain the high-end caramel pigment C.

Some embodiments of the present disclosure further disclose a method for utilizing corn cobs to jointly produce a premium xylose and a high-end caramel pigment, the method using the system for utilizing corn cobs to jointly produce a premium xylose and a high-end caramel pigment. The method may include:
Step 1, transporting corn cob powder stored in the raw material tank to the pretreatment tank through a pipeline for water washing and acid washing to obtain a corn cob liquid, and then sending the corn cob liquid to the acid hydrolysis kettle through a pipeline to perform acid hydrolysis treatment;
Step 2, obtaining a plate and frame filter press feed liquid H after the acid hydrolysis treatment, and sending the plate and frame filter press feed liquid H to the plate and frame filter press device 4 through a pipeline for filtration treatment;
Step 3, filtering the plate and frame filter press feed liquid H by the plate and frame filter press device 4 to obtain a liquid part and a solid part, respectively; wherein the liquid part is the corn cob hydrolysate D, and the solid part is the corn cob waste residue E;
Step 4, performing neutralization treatment of the filtrate neutralization tank 5, decolorization treatment of the nanofiltration membrane separator 6, desalination treatment of the electrodialysis separator 7, concentration treatment of the evaporation concentration tank 8, crystallization treatment of the crystallization tank 9, and solid-liquid separation treatment of the centrifugal separator 10 on the corn cob hydrolysate D to obtain a crystallized xylose and a xylose mother liquid, performing drying treatment of the dryer 11 on the crystallized xylose to obtain a crystal finished product of the premium xylose B, and transporting the xylose mother liquid F to the sugar liquid mixing tank 14 through a pipeline;
the purity of the crystal of the premium xylose B is larger than 99%, and a light transmittance may be larger than 98%; and
Step 5, transporting the corn cob waste residue E sequentially to the filter residue neutralization tank 12 for neutralization treatment and to the enzymatic hydrolysis reaction kettle 13 for enzymatic hydrolysis treatment to obtain the enzymatic solution G, transporting the enzymatic solution G to the sugar liquid mixing tank 14 through a pipeline to be mixed with the xylose mother liquid F to obtain a mixture solution, and then transporting the mixture solution through browning reaction treatment of the browning reaction kettle 15, evaporation treatment of the flash tank 16, separation treatment of the ultrafiltration membrane separator 17, and wiped film evaporation treatment of the wiped film evaporator 18 to obtain a product of the high-end caramel pigment C; wherein a color ratio of the high-end caramel pigment C is as high as 50000 EBC. In some embodiments, the color ratio of high-end caramel pigment C may be as high as any one of 35,000 EBC, 40,000 EBC, 45,000 EBC, and 55,000 EBC.

In some embodiments, in Step 1, a mass percentage concentration of dry sugar resources in the corn cob A may be in a range of 60˜70 wt %, wherein the dry sugar resources may contain hexose sugars of 50˜55 wt % and pentose sugars of 45˜50 wt %, a sulfuric acid solution of 0.20˜0.30 wt % may be used for the acid washing, and a liquid-to-solid ratio for the acid washing may be 3:1. For example, the mass percentage concentration of the dry sugar resources in the corn cob A may be 60 wt %, 65 wt %, 70 wt %, etc., dry sugar resources may contain hexose sugars of 50 wt %, 53 wt %, 55 wt %, etc., and pentose sugars of 45 wt %, 48 wt %, 50 wt %, etc., and the sulfuric acid solution used for the acid washing may be 0.20 wt %, 0.25 wt %, 0.30 wt %, etc.

In some embodiments, in Step 2, during the acid hydrolysis treatment, a ratio of corn cob material to liquid may be 1:5, an added sulfuric acid content may be in a range of 1.2˜1.5%, a temperature may be in a range of 120˜125° C., and a time may be in a range of 100˜140 min. For example, during the acid hydrolysis treatment, the added sulfuric acid content may be 1.2%, 1.3%, etc.; the temperature may be 120° C., 123° C., etc., and the time may be 100 min, 120 min, etc.

In some embodiments, in Step 4, during the decolorization treatment of the nanofiltration membrane separator 6, an operating temperature of the nanofiltration membrane separator 6 may be in a range of 40˜60° C., and an operating pressure may be in a range of 15˜35 bar; during the desalination treatment of the electrodialysis separator 7, an operating current of the electrodialysis separator 7 may be in a range of 60˜120 A, a voltage may be in a range of 200˜250 V, and an operating pressure may be in a range of 15˜35 bar. For example, during the decolorization treatment of the nanofiltration membrane separator 6, the operating temperature may be 40° C., 55° C., 60° C., etc., the operating pressure may be 15 bar, 30 bar, 35 bar, etc. As another example, during the desalination treatment of the electrodialysis separator 7, the operating current of the electrodialysis separator 7 may be 60 A, 100 A, 120 A, etc., a voltage may be 200 V, 220 V, 250 V, etc., and the operating pressure may be 15 bar, 20 bar, 35 bar, etc.

In some embodiments, in Step 5, during the enzymatic hydrolysis treatment, the pH may be adjusted to 5.5˜6.5, a commercial cellulose composite enzyme with a percentage of 2˜4% may be used as a catalyst, an enzymatic reaction temperature may be controlled in a range of 40˜60° C., and an enzymatic reaction time may be in a range of 72˜96 h; during the browning reaction treatment of the browning reaction kettle 15, a reaction auxiliary agent may be a compound of ammonia water or ammonium carbonate, ammonium bicarbonate, and urea, a compound ratio may be in a range of 6:2:2˜4:4:2. A reaction process may include: first adjusting the pH to 7.0˜9.0, then raising the temperature to 80˜90° C. to react for 30˜50 min, then raising the temperature to 160˜180° C. for 3˜5 h, then adjusting the pH to 2˜4, then lowering the temperature to 130˜150° C. to react for 0.5˜1 h. During the separation treatment of the ultrafiltration membrane separator 17, an operating temperature of the ultrafiltration membrane separator 17 may be in a range of 40˜60° C., and an operating pressure may be in a range of 5˜7 bar; and during wiped film evaporation treatment of the wiped film evaporator 18, an evaporation pressure may be in a range of 4˜5 bar, and a vacuum degree may be in a range of 80˜90 kpa.

In some embodiments, during the enzymatic hydrolysis treatment, the pH may be adjusted to 5.5, a commercial cellulose composite enzyme with a percentage of 2% may be added as a catalyst, the enzymatic reaction temperature may be controlled at 40° C., and the enzymatic reaction time may be 72 h; during the browning reaction treatment of browning reaction kettle 15, the reaction auxiliary agent may be a compound of ammonia water or ammonium carbonate, ammonium bicarbonate, and urea, the compound ratio may be 6:2:2. The reaction process may include: first adjusting the pH to 7.0, then raising the temperature to 80° C. to react for 30 minutes, then raising the temperature to 160° C. to react for 3 hours, then adjusting the pH to 2, and then lowering the temperature to 130° C. to react for 0.5 h. During the separation treatment of the ultrafiltration membrane separator 17, the operating temperature of the ultrafiltration membrane separator 17 may be 40° C. and the operating pressure may be 5 bar; and during the wiped film evaporation treatment of the wiped film evaporator 18, the evaporation pressure may be 4 bar, and the vacuum degree may be 80 kPa.

In some embodiments, during the enzymatic treatment, the pH may be adjusted to 6.0, a commercial cellulose complex enzyme with a percentage of 3% may be added as a catalyst, and the temperature of the enzymatic reaction may be controlled to be 50° C., and the enzymatic reaction time may be 85 h; during the browning reaction treatment of browning reaction kettle 15, the reaction auxiliary agent may be a compound of ammonia or ammonium carbonate, ammonium bicarbonate, and urea, the compound ratio may be 5:5:2. The reaction process may include: first adjusting the pH to 8.0, then raising the temperature to 85° C. to react for 40 minutes, then raising the temperature to 170° C. to react for 4 hours, then adjusting the pH to 3, and then lowering the temperature to 140° C. to react for 0.75 h. During the separation treatment of the ultrafiltration membrane separator 17, the operating temperature of the ultrafiltration membrane separator 17 may be 50° C. and the operating pressure may be 6 bar; and during the wiped film evaporation treatment of the wiped film evaporator 18, the evaporation pressure may be 4.5 bar, and the vacuum degree may be 85 kpa.

In some embodiments, during the enzymatic hydrolysis treatment, the pH may be adjusted to 6.5, and the commercial cellulose composite enzyme with a percentage of 4% may be added as a catalyst, the enzymatic reaction temperature may be controlled at 60° C., and the enzymatic reaction time may be 96 h; during the browning reaction treatment of the browning reaction kettle 15, the reaction auxiliary agent may be a compound of ammonia water or ammonium carbonate, ammonium bicarbonate, and urea, the compound ratio may be 4:4:2. The reaction process may include: first adjusting the pH to 9.0, and then raising the temperature to 90° C. to react for 50 min, then raising the temperature to 180° C. to react for 5 h, then adjusting the pH to 4, then lowering the temperature to 150° C. to react for 1 h. During the separation treatment of the ultrafiltration membrane separator 17, the operating temperature of the ultrafiltration membrane separator 17 may be 60° C., and the operating pressure may be 5 bar; and during the wiped film evaporation treatment of the wiped film evaporator 18, the evaporation pressure may be 5 bar, and the vacuum degree may be 90 kpa.

In processes for utilizing corn cobs to jointly produce a premium xylose and a high-end caramel pigment using the corn cobs as a raw material, after purifying the premium xylose, high-end caramel pigments may be prepared by using an enzymatic solution after enzymatic hydrolysis and mixing with a xylose mother liquid after centrifugation according to following steps:
Step 1, transporting the corn cob powder stored in the raw material tank 1 to the pretreatment tank through a pipeline for water washing and acid washing to obtain a corn cob liquid, using a sulfuric acid solution of 0.20 wt % with a liquid-to-solid ratio of 3:1 for the acid washing, then sending the corn cob liquid to the acid hydrolysis kettle 3 to perform acid hydrolysis treatment;
Step 2, transporting a post-wash liquid of the corn cobs in the pretreatment tank 2 to the acid hydrolysis kettle 3 through a pipeline for hydrolysis treatment, with a corn cob liquid-to-solid ratio of 5:1, an added sulfuric acid content of 1.2%, a temperature of 125° C., and a time of 140 min, then sending a hydrolyzed treated material to the plate and frame filter press device 4 for filtration treatment;
Step 3, dividing the hydrolyzed treated material into two parts by the plate and frame filter press device 4, a liquid part being the corn cob hydrolysate D, and a solid part being the corn cob waste residue E;
Step 4, performing neutralization treatment of the filtrate neutralization tank 5, decolorization treatment of the nanofiltration membrane separator 6, desalination treatment of the electrodialysis separator 7, concentration treatment of the evaporation concentration tank 8, crystallization treatment of the crystallization tank 9, and solid-liquid separation treatment of the centrifugal separator 10 on the corn cob hydrolysate D to obtain a crystallized xylose and a xylose mother liquid F, performing drying treatment of the dryer 11 on the crystallized xylose to obtain a crystal finished product of the premium xylose B, transporting the xylose mother liquid F to the sugar liquid mixing tank 14 through a pipeline;
the purity of the crystal of the premium xylose may be larger than 99%, and a light transmittance may be larger than 98%; the operating temperature of the nanofiltration membrane separator 6 may be 60° C., and the operating pressure may be 20 bar; and the operating current of the electrodialysis separator 7 may be 100 A, the voltage may be 250 V, and the operating pressure may be 20 bar;
Step 5, transporting the corn cob waste residue sequentially to the filter residue neutralization tank 12 for neutralization treatment and to the enzymatic hydrolysis reaction kettle 13 for enzymatic hydrolysis treatment to obtain the enzymatic solution G, transporting the enzymatic solution G to the sugar liquid mixing tank 14 through a pipeline to be mixed with the xylose mother liquid F to obtain a mixture solution, and then transporting the mixture solution through the browning reaction treatment of the browning reaction kettle 15, the evaporation treatment of the flash tank 16, the separation treatment of the ultrafiltration membrane separator 17, and the wiped film evaporation treatment of the wiped film evaporator 18 to obtain a product of the high-end caramel pigment C, wherein the color ratio of the high-end caramel pigment C is as high as 50,000 EBC, all other indexes are all compounded with the national standard, and a utilization rate of sugar resources is as high as 90%.

In some embodiments of the present disclosure, the high-end caramel pigment may be prepared by mixing the corn cob waste residue and the pentose and hexose sugars from the xylose mother liquid and the corn cob waste residue, which greatly improves the utilization rate of corn cob resources. At the same time, products of the high-end caramel pigment with a color ratio as high as 50,000 EBC have been prepared, which greatly improves the comprehensive utilization value of corn cobs.

In some embodiments, during the enzymatic hydrolysis treatment, the pH may be adjusted to 5.5, and a commercial cellulose composite enzyme with a percentage of 2% may be added as a catalyst, the enzymatic reaction temperature may be controlled at 50° C., and the enzymatic reaction time may be 72 h.

In some embodiments, during the browning reaction treatment of the browning reaction kettle 15, the reaction auxiliary agent may be a compound of ammonia water or ammonium carbonate, ammonium bicarbonate, and urea, a compound ratio may be 6:2:2. The reaction process may include: first adjusting the pH to 9.0, and then raising the temperature to 90° C. to react for 30 min, then raising the temperature to 170° C. to react for 3 h, then adjusting the pH to 3, then lowering the temperature to 130° C. to react for 1 h. The operating temperature of the ultrafiltration membrane separator 17 may be 60° C., and an operating pressure may be 5 bar. During the wiped film evaporation treatment of the wiped film evaporator 18, the evaporation pressure may be 4 bar, and the vacuum degree may be 90 kpa.

### Comparative Example 1

The corn cob A was used as a raw material for the direct extraction of xylose. The extraction process includes: transporting the raw material of the corn cob in the raw material tank 1 through pipelines to the pretreatment tank 2 for washing, transporting the washed raw material to the acid hydrolysis kettle 3, adding 1.5% sulfuric acid, and then performing alkali neutralization, activated carbon decolorization, ion exchange desalination by passing the plate and frame filter press device 4 on the hydrolysate, and then performing evaporation concentration, crystallization, and centrifugation to finally obtain a finished product crystallized xylose. After the plate and frame press filtration, the corn cob waste residue E was dried and burned or treated as solid waste, and the xylose mother liquid F after centrifugation was sold as syrup, and the actual utilization rate of sugar resources was only 30%.

### Comparative Example 2

The caramel pigment was directly prepared using the xylose mother liquid F as a raw material, and the specific process include: transporting the xylose mother liquid F after centrifugal separation directly to the browning reaction kettle 15 through a pipeline, adjusting the pH to 9.0, then adding 10 w % ammonium carbonate as a reaction aid, wherein the reaction temperature was 170° C., and the reaction time was 3 hours. After the reaction, the finished product of caramel pigment was obtained by filtration. The color ratio of the caramel pigment was 30,000 EBC, and all other indexes were in accordance with the national standard.

In summary, using the system and method for utilizing corn cobs to jointly produce a premium xylose and a high-end caramel pigment of some embodiments of the present disclosure, the utilization rate of the sugar resources in the corn cob A is increased from 30% to 90%, and the color ratio of the caramel pigment is increased from 30,000 EBC to 50,000 EBC, which improves the comprehensive utilization value of the corn cob and also improves the product quality of the caramel pigment. Through the improvement of the whole process system and the optimization of the caramel pigment preparation process, a plurality of hexose sugars in the corn cob waste residue and the pentose sugars in the xylose mother liquid are prepared into high-end caramel pigment, which improves the quality of the caramel pigment product. By converting the xylose mother liquid and corn cob waste residue priced at 1,000 yuan/ton into high-end caramel pigments priced at 5,000 yuan/ton, the economic benefits can be increased by more than 5 times. The burning or large accumulation of corn cob waste occupies environmental resources and affects the construction of ecological civilization. The comprehensive utilization of corn cob waste is conducive to sustainable development and the construction of green ecological civilization.

In some embodiments, the system for utilizing corn cobs to jointly produce a premium xylose and a high-end caramel pigment may further include a pH monitoring device, a charging device, a temperature monitoring device, and a processor. The charging device may be connected to the acid hydrolysis kettle. In some embodiments, the pH monitoring device, the charging device, and the temperature monitoring device may be communicatively connected to the processor.

In some embodiments, the pH monitoring device may be deployed within the acid hydrolysis kettle and configured to obtain pH monitoring data of the acid hydrolysis kettle. The pH monitoring data refers to data related to a pH value of an acid liquid in the acid hydrolysis kettle.

The charging device may be a device for adding an acid liquid to the acid hydrolysis kettle. The charging device may include an intelligent quantitative doser, a motorized pipette, etc.

In some embodiments, the temperature monitoring device may be deployed within the acid hydrolysis kettle and configured to obtain the temperature data of the acid hydrolysis kettle.

In some embodiments, the processor may be configured to generate a charging control instruction based on the pH monitoring data and send the charging control instruction to the charging device to control the charging device to add the acid liquid to the acid hydrolysis kettle.

The charging control instruction may control the charging device to add the acid liquid to the acid hydrolysis kettle based on preset acid dosing parameters. The preset acid dosing parameters may include data related to a pH value of the added acid liquid, a volume of the acid liquid, and an acid addition time.

The acid addition time refers to a time satisfying an acid hydrolysis addition condition. The acid hydrolysis addition condition may include the pH monitoring data exceeding a pH threshold.

In some embodiments, if the pH monitoring data exceeds the pH threshold, the processor may determine that a current addition amount is not sufficient to completely hydrolyze the material in the acid hydrolysis kettle 3. At this point, the processor may generate a charging control instruction to control the charging device to add acid liquid to the acid hydrolysis kettle 3 based on the preset acid dosing parameters until the pH value no longer changes and is less than the pH threshold, thereby achieving complete hydrolysis.

In some embodiments, the processor may determine the pH threshold by performing a vector search in a first vector database based on temperature data, initial pH data, and corn cob composition data.

The initial pH data refers to pH-related data after the corn cob liquid is added to the acid hydrolysis kettle 3 and before the acid liquid is added. The processor may obtain the initial pH data by accessing historical data stored in a storage unit.

The corn cob composition data refers to a content of sugars in the corn cob and other composition-related data, for example, the content of hexose sugars, pentose sugars, or the like.

In some embodiments, the processor may obtain the corn cob composition data of the same batch of corn cobs by sampling and testing the same batch of corn cobs and input the corn cob composition data into the storage unit through any feasible means, such as obtaining input data from a user terminal.

In some embodiments, the processor may construct a first vector to be matched based on the temperature data, the initial pH data, and the corn cob composition data.

The first vector database may include a plurality of first reference vectors, and each of the first reference vectors has a correspondence with the pH threshold. The pH threshold corresponding to the first reference vectors may be obtained based on historical data, actual experience, or the like. The first reference vector may be constructed in a similar manner to the first vector to be matched.

In some embodiments, the processor may determine the pH threshold based on a similarity between the first to-be-matched vector and the plurality of first reference vectors in the first vector database. For example, a first reference vector whose similarity with the first to-be-matched vector satisfies a preset condition may be taken as a first target vector, and a pH threshold corresponding to the first target vector may be taken as a finalized pH threshold. The preset condition may be set according to the situation. For example, the preset condition may be that the similarity is maximum, the similarity is greater than a threshold, etc.

In some embodiments of the present disclosure, by monitoring the pH value of the acid hydrolysis kettle after adding the acid liquid, the pH value is accurately controlled in real time, and the degree of the reaction is judged, which helps to ensure that a sufficient amount of acid liquid is added for the acid washing, ensuring a complete reaction while saving acid, and improving the degree of acid hydrolysis of the corn cob powder.

In some embodiments, a stirring device may be disposed in the pretreatment tank, the acid hydrolysis kettle, the filtrate neutralization tank, and the filter residue neutralization tank, respectively.

In some embodiments, the processor may generate a stirring control instruction and send the stirring control instruction to the stirring device to control a stirring power of the stirring device.

In some embodiments, the processor may determine the stirring power based on a total weight of materials and additives within each of the treatment tank 2, the acid hydrolysis kettle 3, the filtrate neutralization tank 5, and the filter residue neutralization tank 12. For example, the greater the total weight of the materials and additives within each device, the greater the stirring power.

The total weight of the materials and additives may be obtained by sensors provided in each device and uploaded to the storage unit, which is then recalled by the processor in real-time. More description of the storage unit may be found in a later description.

Exemplarily, the material in the pretreatment tank 2 is corn cob powder, and the additive is a sulfuric acid solution of 0.20˜0.30 wt %; the total weight will increase after the addition of the acid liquid, and at this time, the stirring power may be increased accordingly.

In some embodiments of the present disclosure, by timely adjusting the stirring power in the stirring device, the reaction condition may be quickly updated, effectively accelerating the reaction rate.

In some embodiments, the system for utilizing corn cobs to jointly produce a premium xylose and a high-end caramel pigment may further include a temperature control device and a storage unit. The temperature control device may include a plurality of heating plates and heating unit power supply and may be connected to a circuit of the processor.

The plurality of heating plates may be deployed at preset intervals around outside and a bottom of the acid hydrolysis kettle, and the plurality of heating plates of the temperature control device may be configured to heat a mixed solution of the corn cob liquid and the acid liquid in the acid hydrolysis kettle by energizing the temperature.

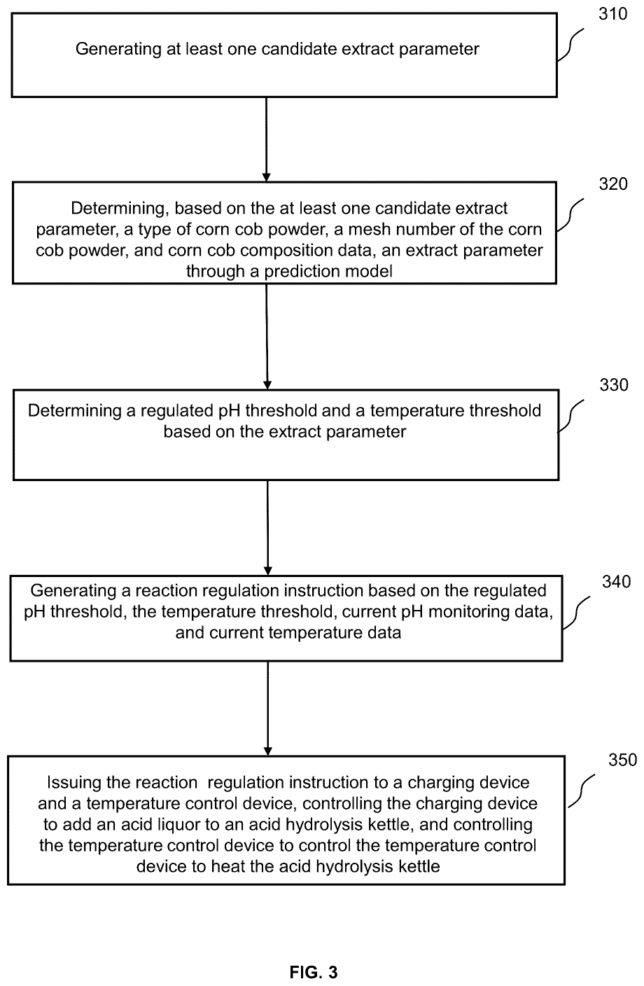

As shown in FIG. 3, the processor may realize controlling processes performed by the charging device and the temperature control device on the acid hydrolysis kettle by operations 310 to 350.

In 310, as least one candidate extraction parameter may be generated.

The candidate extraction parameter refers to a parameter in the acid hydrolysis kettle that is related to the extraction reaction of the corn cob. For example, the candidate extraction parameter may be a candidate regulated pH range, a candidate hydrolysis temperature, or the like.

In some embodiments, the processor may determine the candidate extraction parameter by random generation or other feasible means.

In 320, an extraction parameter may be determined based on at least one candidate extraction parameter, a type of corn cob powder, a mesh number of the corn cob powder, and the corn cob composition data through a prediction model. The extraction parameter may include a regulated pH range and a temperature range.

In some embodiments, the prediction model may be a machine learning model, e.g., a recurrent neural network (RNN).

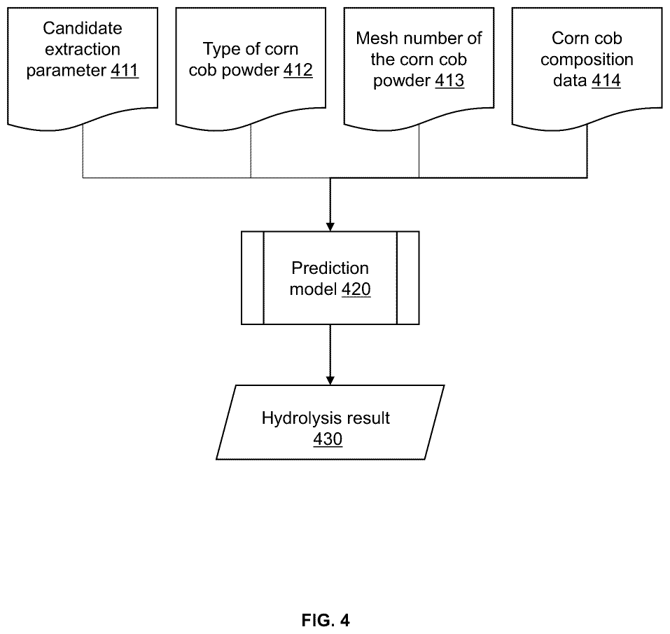

As shown in FIG. 4, an input of a prediction model 420 may include a candidate extraction parameter 411, a type of corn cob powder 412, a mesh number of the corn cob powder 413, and corn cob composition data 414, and an output of the prediction model 420 may include a hydrolysis result 430.

The related descriptions of the corn cob composition data may be found in the corresponding previous descriptions. The candidate extraction parameter, the type of the corn cob powder, the mesh number of the corn cob powder, and the corn cob composition data may be obtained by calling historical data of the storage unit or any other feasible means.

The prediction model may be trained by a remote server based on training samples and transferred to the storage unit. The training samples may be stored in the storage unit and/or the remote server.

The prediction model may be obtained by training based on a large number of training samples with labels. For example, the processor may input a plurality of training samples with labels into an initial prediction model, construct a loss function based on the labels and the results of the initial prediction model, and iteratively, based on the loss function, update the parameters of the initial prediction model by gradient descent or other manners. The model training may be completed when a preset condition is met, and a trained prediction model may be obtained. The preset condition may be that the loss function converges, a count of iterations reaches a threshold, etc.

In some embodiments, the training samples for the prediction model may include a sample extraction parameter, a type of sample corn cob powder, a mesh number of the sample corn cob powder, and sample corn cob composition data, and the labels may characterize an actual hydrolysis result corresponding to the training samples. The training samples may be obtained based on the storage unit and/or the remote server. The label may be generated based on hydrolysis results obtained from the line or manually labeled.

In some embodiments of the present disclosure, the hydrolysis result is predicted by the prediction model, and a candidate extraction parameter with a better hydrolysis result may be further selected as a determined extraction parameter, so that a suitable extraction parameter may be obtained with high efficiency, which may help to improve the corn cob liquid's hydrolysis efficiency.

In some embodiments, the processor may also obtain the hydrolysis result by other feasible means. In some embodiments, the processor may obtain the hydrolysis result based on a hydrolysis completion time and an average pH value.

The hydrolysis completion time refers to a time when the corn cob liquid is actually hydrolyzed using the extraction parameter so that the corn cob liquid in the acid hydrolysis kettle 3 reaches a sufficient degree of hydrolysis.

The average pH value refers to an average pH value during hydrolysis.

The hydrolysis result may be negatively correlated with the hydrolysis completion time and positively correlated with the average pH value.

Exemplarily, the hydrolysis result may be obtained through equation (1):

Hydrolysis result=factor 1*hydrolysis completion time+factor 2*average pH value (1)

As a result of striving to achieve a better hydrolysis rate by using as little acid liquid as possible, the shorter the hydrolysis completion time and the higher the average pH value, the better the hydrolysis result may be, the coefficient 1 is a negative number, and the coefficient 2 is a positive number.

In some embodiments, the processor may select a candidate extraction parameter with the best hydrolysis result as the determined extraction parameter.

In 330, a regulated pH threshold and a temperature threshold may be determined based on the extraction parameter.

Since the acid hydrolysis reaction is an endothermic reaction, the actual pH value will increase and the temperature will decrease during the acid hydrolysis process if no additional heating and acid addition are performed.

Therefore, in some embodiments, the processor may determine the regulated pH threshold and the temperature threshold based on the regulated pH range and the target temperature range of the extraction parameters.

For example, the processor may set an upper limit of the regulated pH range, a target temperature range, and a lower limit of the parameter as the regulated pH threshold and the temperature threshold, respectively.

For example, if the regulated pH range is from 3.0 to 4.0, and the target hydrolysis temperature is in a range of 120-125° C., then the corresponding regulated pH threshold may be equal to or slightly less than 4.0, and the temperature threshold may be equal to or slightly above 120° C.

In 340, a reaction regulation instruction may be generated based on the regulated pH threshold, the temperature threshold, current pH monitoring data, and current temperature data.

In some embodiments, the processor may determine whether the current pH value and the current temperature are appropriate based on the regulated pH threshold and the temperature threshold. When the current pH value is higher than the regulated pH threshold and the current temperature is lower than the temperature threshold, the processor may control the addition of additional acid liquid or performs heating to achieve regulation of the pH value and temperature in the acid hydrolysis kettle 3.

For example, if the current temperature data is below the temperature threshold, the processor may generate a reaction regulation instruction to control the temperature control device to ramp up the temperature of the acid hydrolysis kettle 3 until a target temperature range is reached, for example, reaching the upper limit of the target temperature range.

In 350, the reaction regulation instruction may be issued to the charging device and the temperature control device to control the charging device to add the acid liquid to the acid hydrolysis kettle, and control the temperature control device to heat the acid hydrolysis kettle.

In some embodiments of the present disclosure, by setting up the temperature control device including the plurality of heating plates, temperature control may be carried out modularly to achieve effective temperature control and prevent uneven heating of raw materials from affecting hydrolysis results; and by determining the extraction parameter and generating the reaction regulation instruction, accurate and efficient control of the charging device to add acid liquid to the acid hydrolysis kettle may be realized.

In some embodiments, in response to the pH monitoring data exceeding the regulated pH threshold, the processor may determine an additional addition amount of acid liquid based on the current pH monitoring data, the current temperature data, a weight of raw material, and a historical addition amount of acid liquid in a number of ways. The additional addition amount of acid liquid refers to an amount of acid liquid that, when added with a corresponding amount of acid liquid, may allow the pH value in the hydrolysis kettle 3 to be adjusted to the regulated pH range, or to be adjusted to a specific value within the regulated pH range, such as allowing the pH value to be just reduced to the lower limit of 3.0 of the regulated PH range.

In some embodiments, the processor may construct a regulation feature vector based on the current monitoring data, acid parameters, a historical material-liquid ratio, and the regulated pH range, and determine the additional addition amount of acid liquid by conducting vector searches in a second vector database.

The current monitoring data may include the current pH value and the current temperature data.

The acid parameters may include an acid concentration, e.g., a concentration of sulfuric acid.

The historical material-liquid ratio refers to a weight of the raw material (e.g., the corn cob liquid) and a historical addition amount of acid liquid.

The second vector database may include a plurality of second reference vectors, and each of the second reference vectors has a correspondence with the additional addition amount of acid liquid. The additional addition amount of acid liquid may be obtained based on historical data, actual experience, or the like.

The processor may construct the second reference vector based on historical monitoring data, historical acid parameters, a historical material-liquid ratio, and a historical regulated pH range. The second reference vector may be constructed in a similar manner as the vector to be matched.

In some embodiments, the processor may determine the additional addition amount of acid liquid based on similarities between a second vector to be matched and a plurality of second reference vectors in the second vector database. For example, a second reference vector whose similarity with the second to-be-matched vector satisfies a preset condition may be used as a second target vector, and the additional addition amount of acid liquid corresponding to the second target vector may be used as a finalized additional addition amount of acid liquid. The preset condition may be set according to the situation. For example, the preset condition may be that the similarity is maximum, the similarity is greater than a threshold, etc.

In some embodiments of the present disclosure, by obtaining the amount of additional added acid according to the actual situation, it is possible to determine a reasonable amount of acid liquid to ensure that the pH value of the acid liquid in the acid hydrolysis kettle is in a range of pH values that are most suitable for the hydrolysis reaction, and at the same time, it is possible to effectively avoid the waste of acid liquid, which is conducive to the realization of cost reduction and efficiency increase of the process flow.

In some embodiments, the system for utilizing corn cobs to jointly produce a premium xylose and a high-end caramel pigment may further include a filtrate temporary storage tank and a filtrate residue temporary storage tank.

In some embodiments, an input port of the filtrate temporary storage tank may be connected to the filtrate outlet of the plate and frame filter press device; and an output port of the filtrate temporary storage tank may be connected to the filtrate neutralization tank.

In some embodiments, the input port of the filtrate residue temporary storage tank may be connected to the filter residue outlet of the plate and frame filter press device.

In some embodiments, an output port of the filtrate residue temporary storage tank may be connected to the feed port of the filter residue neutralization tank. The filter residue temporary storage tank may be further configured with a filter residue weighing device, the filter residue weighing device being used to obtain a weight of filter residue entering the filter residue temporary storage tank.

In some embodiments, the processor may determine whether to inspect the system for utilizing corn cobs to jointly produce a premium xylose and a high-end caramel pigment based on the weight of filter residue and a total amount of plate and frame filter press feed liquid.

In some embodiments, the processor may determine an estimated weight of filter residue based on the total amount of plate and frame filter press feed liquid by querying a preset table. The preset table may include the total amount of the plate and frame filter press feed liquid as well as a historical weight of filter residue. The processor may obtain the preset table by recalling historical data from the storage unit.

In some embodiments, the processor may determine, by obtaining the weight of filter residue, whether to inspect the system, e.g., to inspect the plate and frame filter press device 4 and the filtrate.

In some embodiments, if a difference between the estimated weight of filter residue and an actual weight of filter residue is greater than a predetermined threshold, the processor may determine that there is a malfunction in the plate and frame filter press device 4, which needs to be inspected and repaired.

When the repair is complete, the processor may generate a control instruction to control the filtrate to be re-entered into the plate and frame filter press device 4 for filter press.

In some embodiments, if the difference between the estimated weight of filter residue and the actual weight of filter residue is less than or equal to the predetermined threshold, subsequent operations of the current process may continue.

In some embodiments of the present disclosure, by obtaining the weight of filter residue and determining whether to inspect the system, in particular, the plate and frame filter press device and the filtrate, the malfunctioning and the clogging of the filter press device can be effectively avoided, and the filter residue can be ensured to be more thoroughly removed by filtration.

The foregoing is only preferred embodiments of some embodiments of the present disclosure, and is not intended to limit some embodiments of the present disclosure. Any modifications, equivalent substitutions, and improvements, etc., within the spirit and principles of some embodiments of the present disclosure shall be included in the scope of protection of some embodiments of the present disclosure.

What is claimed is:

1. A system for utilizing corn cobs to jointly produce a premium xylose and a high-end caramel pigment, comprising a xylose preparation subsystem, an enzymatic hydrolysis subsystem, and a caramel pigment preparation subsystem that are connected to each other through pipelines; wherein
the xylose preparation subsystem includes a raw material tank, a pretreatment tank, an acid hydrolysis kettle, a plate and frame filter press device, a filtrate neutralization tank, a nanofiltration membrane separator, an electrodialysis separator, an evaporation concentration tank, a crystallization tank, a centrifugal separator, and a dryer that are connected in sequence through pipelines;
the enzymatic hydrolysis subsystem includes a filter residue neutralization tank and an enzymatic hydrolysis reaction kettle connected through pipelines;
the caramel pigment preparation subsystem includes a sugar liquid mixing tank, a browning reaction kettle, a flash tank, an ultrafiltration membrane separator, and a wiped film evaporator connected in sequence through pipelines; and
the plate and frame filter press device is provided with a filtrate outlet and a filter residue outlet; the filtrate outlet is connected to the filtrate neutralization tank through a pipeline; the filter residue outlet is connected to a feed port of the filter residue neutralization tank through a pipeline, the centrifugal separator is provided with a solid outlet and a liquid outlet, the solid outlet is connected to a feed port of the dryer through a pipeline, and the liquid outlet is connected to one feed port of the sugar liquid mixing tank, the other feed port of the sugar liquid mixing tank is connected to a discharge port of the enzymatic hydrolysis reaction kettle through a pipeline, the raw material tank stores a raw material to be processed of the corn cobs, a material output by a discharge port of the dryer is the premium xylose, and a final material obtained from a discharge port of the wiped film evaporator is the high-end caramel pigment.

2. The system of claim 1, wherein a filtrate output from a filtrate outlet of the plate and frame filter press device is a corn cob hydrolysate, and a filter residue output from a filter residue outlet of the plate and frame filter press device is a corn cob waste residue.

3. The system of claim 1, wherein a liquid material transported from a liquid outlet of the centrifugal separator is a xylose mother liquid.

4. The system of claim 1, wherein a liquid output from the discharge port of the enzymatic hydrolysis reaction kettle is an enzymatic solution.

5. The system of claim 1, further comprising a pH monitoring device, a charging device, a temperature monitoring device, and a processor, wherein
the charging device is connected to the acid hydrolysis kettle;
the pH monitoring device is deployed in the acid hydrolysis kettle and configured to obtain PH monitoring data of the acid hydrolysis kettle;
the temperature monitoring device is deployed in the acid hydrolysis kettle and configured to obtain temperature data of the acid hydrolysis kettle; and
the processor is configured to:
generate a charging control instruction based on the pH monitoring data; and
send the charging control instruction to the charging device to control the charging device to add an acid liquid to the acid hydrolysis kettle.

6. The system of claim 5, wherein a stirring device is disposed in the pretreatment tank, the acid hydrolysis kettle, the filtrate neutralization tank, and the filter residue neutralization tank, respectively;
the processor is further configured to generate a stirring control instruction and send the stirring control instruction to the stirring device to control a stirring power of the stirring device.

7. The system of claim 5, further comprising a temperature control device and a storage unit, wherein the temperature control device includes a plurality of heating plates and heating unit power supply, the temperature control device is connected to a circuit of the processor, the plurality of heating plates are deployed at preset intervals around outside and at a bottom portion of the acid hydrolysis kettle and are configured to increase a temperature by energizing to heat a mixed solution of a corn cob liquid and the acid liquid in the acid hydrolysis kettle; and
the processor is further configured to:
generate at least one candidate extraction parameter;
determine, based on the at least one candidate extraction parameter, a type of corn cob powder, a mesh number of the corn cob powder, and corn cob composition data, an extraction parameter through a prediction model; the prediction model being a machine learning model, and the prediction model being obtained by a remote server based on a training sample and transmitted to the storage unit, the training sample being stored in the storage unit and/or the remote server; the extraction parameter including a regulated pH range and a target temperature range;
determine a regulated pH threshold and a temperature threshold based on the extraction parameter;
generate a reaction regulation instruction based on the regulated pH threshold, the temperature threshold, current pH monitoring data, and current temperature data; and
issue the reaction regulation instruction to the charging device and the temperature control device, control the charging device to add the acid liquid to the acid hydrolysis kettle, and control the temperature control device to heat the acid hydrolysis kettle.

8. The system of claim 7, wherein in response to the pH monitoring data being higher than the regulated pH threshold, the processor is further configured to:
determine, based on the current pH monitoring data, the current temperature data, a weight of raw materials, and a historical addition amount of acid liquid, an additional addition amount of acid liquid.

9. The system of claim 1, further comprising a filtrate temporary storage tank and a filter residue temporary storage tank; wherein
an input port of the filtrate temporary storage tank is connected to a filtrate outlet of the plate and frame filter press device; and an output port of the filtrate temporary storage tank is connected to the filtrate neutralization tank;
an input port of the filter residue temporary storage tank is connected to a filter residue outlet of the plate and frame filter press device;
an output port of the filter residue temporary storage tank is connected to the feed port of the filter residue neutralization tank, the filter residue temporary storage tank is also provided with a filter residue weighing device, the filter residue weighing device is configured to obtain a weight of filter residue entering the filter residue temporary storage tank; and
the processor is further configured to: determine whether to inspect the system based on the weight of filter residue and a total amount of plate and frame filter press feed liquid.

10. A method for utilizing corn cobs to jointly produce a premium xylose and a high-end caramel pigment using the system of claim 1, comprising:
Step 1: transporting corn cob powder stored in the raw material tank to the pretreatment tank through a pipeline for water washing and acid washing to obtain a corn cob liquid, and then sending the corn cob liquid to the acid hydrolysis kettle through a pipeline to perform acid hydrolysis treatment;
Step 2: obtaining a plate and frame filter press feed liquid after the acid hydrolysis treatment, and sending the plate and frame filter press feed liquid to the plate and frame filter press device through a pipeline for filtration treatment;
Step 3: filtering the plate and frame filter press feed liquid by the plate and frame filter press device to obtain a liquid part and a solid part, respectively; wherein the liquid part is a corn cob hydrolysate, and the solid part is a corn cob waste residue;
Step 4: performing neutralization treatment of the filtrate neutralization tank, decolorization treatment of the nanofiltration membrane separator, desalination treatment of the electrodialysis separator, concentration treatment of the evaporation concentration tank, crystallization treatment of the crystallization tank, and solid-liquid separation treatment of the centrifugal separator on the corn cob hydrolysate to obtain a crystallized xylose and a xylose mother liquid, respectively, performing drying treatment of the dryer on the crystallized xylose to obtain a crystal finished product of a premium xylose, transporting the xylose mother liquid to the sugar liquid mixing tank through a pipeline; wherein a purity of the crystal finished product of the premium xylose is larger than 99%, and a light transmittance of the crystal finished product of the premium xylose is larger than 98%; and
Step 5: transporting the corn cob waste residue sequentially to the filter residue neutralization tank for neutralization treatment and to the enzymatic hydrolysis reaction kettle for enzymatic hydrolysis treatment to obtain an enzymatic solution, transporting the enzymatic solution to the sugar liquid mixing tank through a pipeline to be mixed with the xylose mother liquid to obtain a mixture solution, and then transporting the mixture solution through browning reaction treatment of the browning reaction kettle, evaporation treatment of the flash tank, separation treatment of the ultrafiltration membrane separator, and wiped film evaporation treatment of the wiped film evaporator to obtain a product of the high-end caramel pigment; wherein a color ratio of the high-end caramel pigment is as high as 50000 EBC.

11. The method of claim 10, wherein in Step 1, a mass percentage concentration of dry sugar resources in the corn cobs is in a range of 60˜70 wt %, the dry sugar resources contain hexose sugars of 50˜55 wt % and pentose sugars of 45˜50 wt %; a sulfuric acid solution of 0.20˜0.30 wt % is used for the acid washing, and a liquid-to-solid ratio for the acid washing is 3:1.

12. The method of claim 10, wherein in Step 2, during the acid hydrolysis treatment, a ratio of corn cob material to liquid is 1:5, an added sulfuric acid content is in a range of 1.2˜1.5%, a temperature is in a range of 120˜125° C., and a time is in a range of 100˜140 min.

13. The method of claim 10, wherein in Step 4, during the decolorization treatment of the nanofiltration membrane separator, an operating temperature of the nanofiltration membrane separator is in a range of 40˜60° C., and an operating pressure is in a range of 15˜35 bar; and during the desalination treatment of the electrodialysis separator, an operating current of the electrodialysis separator is in a range of 60˜120 A, a voltage is in a range of 200˜250 V, and an operating pressure is in a range of 15˜35 bar.

14. The method of claim 10, wherein in Step 5, during the enzymatic hydrolysis treatment, pH is adjusted to 5.5˜6.5, and a commercial cellulose composite enzyme with a percentage of 2˜4% is added as a catalyst, an enzymatic hydrolysis reaction temperature is controlled in a range of 40˜60° C., and an enzymatic hydrolysis reaction time is in a range of 72˜96 h; during the browning reaction treatment of the browning reaction kettle, a reaction auxiliary agent is a compound of ammonia water or ammonium carbonate, ammonium bicarbonate, and urea, a compound ratio is in a range of 6:2:2˜4:4:2,
a reaction process includes: first adjusting pH to 7.0˜9.0, and then raising the temperature to 80˜90° C. to react for 30˜50 min, then raising the temperature to 160˜180° C. to react for 3˜5 h, then adjusting the pH to 2˜4, and then lowering the temperature to 130˜150° C. to react for 0.5˜1 h;
during the separation treatment of the ultrafiltration membrane separator, an operating temperature of the ultrafiltration membrane separator is in a range of 40˜60° C., and an operating pressure is in a range of 5˜7 bar; and
during the wiped film evaporation treatment of the wiped film evaporator, an evaporation pressure is in a range of 4˜5 bar, and a vacuum degree is in a range of 80˜90 kpa.

15. The method of claim 10, wherein the method is executed by a processor, and the method further comprises:
obtaining pH monitoring data of the acid hydrolysis kettle through a pH monitoring device;
obtaining temperature data of the acid hydrolysis kettle through a temperature monitoring device;
generating a charging control instruction based on the pH monitoring data; and
sending the charging control instruction to a charging device to control the charging device to add an acid liquid to the acid hydrolysis kettle.

16. The method of claim 15, further comprising:
generating a stirring control instruction, and sending the stirring control instruction to a stirring device to control a stirring power of the stirring device.

17. The method of claim 15, further comprising:
generating at least one candidate extraction parameter;
determining, based on the at least one candidate extraction parameter, a type of corn cob powder, a mesh number of the corn cob powder, and corn cob composition data, an extraction parameter through a prediction model; the prediction model being a machine learning model, and the prediction model being obtained by a remote server based on a training sample and transmitted to a storage unit, the training sample being stored in the storage unit and/or the remote server; the extraction parameter including a regulated pH range and a target temperature range;
determining a regulated pH threshold and a temperature threshold based on the extraction parameter;
generating a reaction regulation instruction based on the regulated pH threshold, the temperature threshold, current pH monitoring data, and current temperature data; and
issuing the reaction regulation instruction to the charging device and a temperature control device, controlling the charging device to add the acid liquid to the acid hydrolysis kettle, and controlling the temperature control device to heat the acid hydrolysis kettle.

18. The method of claim 17, wherein in response to the pH monitoring data being higher than the regulated pH threshold, the generating a reaction regulation instruction based on the regulated pH threshold, the temperature threshold, current pH monitoring data, and current temperature data includes:
determining, based on the current pH monitoring data, the current temperature data, a weight of raw materials, and a historical addition amount of acid liquid, an additional addition amount of acid liquid.

19. The method of claim 10, further comprising:
determine whether to inspect the system based on a weight of filter residue and a total amount of plate and frame filter press feed liquid.
